(19) United States
(12) Patent Application Publication
(10) Pub. No.: US 2026/0115439 A1
(43) Pub. Date: Apr. 30, 2026

(54) MICRONEEDLE PATCH HAVING IMPROVED DEFORMABILITY AND MICRONEEDLE PATCH KIT INCLUDING SAME

(71) Applicant: NICO MEDICAL CO., LTD., Gwangju-si, Gyeonggi-do (KR)

(72) Inventors: Won-Hyoung RYU, Goyang-si, Gyeonggi-do (KR); Seung-Hyun PARK, Busan (KR); Youn-Tak KIM, Namyangju-si, Gyeonggi-do (KR); Sang-IL HYUN, Suwon-si, Gyeonggi-do (KR)

(21) Appl. No.: 18/702,145

(22) PCT Filed: Nov. 30, 2023
(86) PCT No.: PCT/KR2023/019554

§ 371 Date: Apr. 17, 2024

(30) Foreign Application Priority Data
Apr. 27, 2023 (KR) 10-2023-0055635

(51) Int. Cl. A61M 37/00; A61K 9/00

(52) U.S. Cl. CPC A61M 37/0015; A61K 9/0021; A61M 2037/0023; A61M 2037/0061

(57) ABSTRACT

The present disclosure relates to a microneedle patch, wherein the microneedle patch includes a microneedle array, the microneedle array including a central portion and a plurality of peripheral portions connected to the central portion and surrounding the central portion, and the peripheral portion is disposed to be spaced apart from an adjacent peripheral portion, wherein the microneedle array includes a hard planar substrate and a plurality of microneedles disposed on the hard substrate, and the peripheral portion is folded through a gap between the adjacent peripheral portion, and the microneedle patch may be attached even to a curved affected area with high adhesion.

### TECHNICAL FIELD

The present disclosure relates to a microneedle patch having improved deformability and a microneedle patch kit including the same.

### BACKGROUND ART

As skin problems are caused by various factors such as stress, eating habits, changing seasons, wearing masks, and the like, the development of products to alleviate such problems is being conducted. Trouble occurs when excessive secretion of sebum from sebaceous glands inside pores happens, causing the pores to become clogged, bacteria to proliferate therein, inflammation to occur, and a protruding surface like a protrusion is formed on the outside of the skin. Since most of these problems occur on the face, there is increasing demand for treatment methods that are rapid and reliable in terms of beauty, and that may be easily applied by anyone.

There are various types of products for improving skin problems, including skin care, cleansing, patches, and ointments. Thereamong, the patches may include a plurality of microneedles, so that the patches may deliver drugs to local areas and provide skin regeneration effects. However, since most microneedle patches have low flexibility, if the patch is attached to a face which actively moves, the microneedle patches may be easily detached from the skin surface. In addition, when the microneedle patch is attached to a curved area, such as skin problems such as acne, adhesion of the patch decreases, and as only a portion of the microneedles included in the patch may be inserted into the affected area, there may be a problem in which the skin problem treatment effect is significantly reduced.

Accordingly, there is a demand for the development of a microneedle patch that can be attached even to a curved affected area with high adhesion.

### SUMMARY OF INVENTION

### Technical Problem

The present disclosure is intended to solve the problems of the prior art, and an aspect of the present disclosure is to provide a microneedle patch which has improved deformability and can be attached to a curved affected area with high adhesion, and a microneedle patch kit including the same.

Another aspect of the present disclosure is to provide a microneedle patch in which microneedles can be uniformly inserted into a central portion and a peripheral portion of a protruding affected area, and a microneedle kit including the same.

### Solution to Problem

According to an aspect of the present disclosure, a microneedle patch includes a microneedle array including a hard planar substrate, and a plurality of microneedles located on the hard substrate, wherein the microneedle array includes a central portion and a plurality of peripheral portions connected to the central portion and surrounding the central portion, wherein the peripheral portion is disposed to be spaced apart from an adjacent peripheral portion, and is folded through a gap between the adjacent peripheral portion.

The microneedle patch according to the present disclosure may further include a soft substrate disposed below the microneedle array.

In the microneedle patch according to the present disclosure, the gap may be a planar portion having a step difference from the microneedle array.

In the microneedle patch according to the present disclosure, the gap may be formed by exposing an upper surface of the soft substrate.

In the microneedle patch according to the present disclosure, the upper surface of the soft substrate may have adhesion.

In the microneedle patch according to the present disclosure, an area of the peripheral portion may be greater than an area of the gap between the adjacent peripheral portion.

In the microneedle patch according to the present disclosure, an outer peripheral surface of the central portion, in contact with the gap may be rounded.

The microneedle patch according to the present disclosure may further include a protective film disposed on the microneedle patch.

In the microneedle patch according to the present disclosure, the soft substrate may include a hydrocolloid layer.

In the microneedle patch according to the present disclosure, the microneedle array may contain a biocompatible polymer.

In the microneedle patch according to the present disclosure, the microneedle array may contain an active ingredient.

In the microneedle patch according to the present disclosure, the central portion may be in contact with a protruding affected area, and the peripheral portion may surround a peripheral portion of the affected area and be in contact therewith.

The present disclosure includes a microneedle patch including the above-described microneedle patch.

According to another aspect of the present disclosure, a microneedle patch kit includes: a release film; and a plurality of microneedle patches disposed to be spaced apart from each other on the release film.

The microneedle patch kit according to the present disclosure may further include a protective film covering and protecting the release film and the plurality of microneedle patches.

In the microneedle patch kit according to the present disclosure, the release film may include a fracture line formed in one direction.

In the microneedle patch kit according to the present disclosure, a plurality of microneedle patches may be disposed on the fracture line.

In the microneedle patch kit according to the present disclosure, the release film may further include a plurality of microneedle patch receiving portions disposed to be spaced apart from the fracture line, wherein the plurality of microneedle patches may be individually packaged by the microneedle patch receiving portions.

### Advantageous Effects of Invention

As set forth above, according to the present disclosure, a microneedle patch may be attached even to a curved affected area with high adhesion.

In addition, a microneedle patch in which microneedles are uniformly inserted into the central portion and the peripheral portion of the affected area, thereby improving a treatment effect and a microneedle patch kit including the same may be provided.

### BRIEF DESCRIPTION OF DRAWINGS

FIG. 1 is a plan view and side view illustrating a microneedle patch according to a first embodiment of the present disclosure.

FIG. 2 is a plan view and side view illustrating a microneedle patch according to a second embodiment of the present disclosure.

FIG. 3 is a schematic diagram of applying the microneedle patch according to the first embodiment of the present disclosure to a human body.

FIG. 4 is a plan view of the microneedle patch according to third and fourth embodiments of the present disclosure.

FIG. 5 is a plan view illustrating a microneedle patch kit according to an embodiment of the present disclosure.

FIG. 6 is a plan view illustrating a microneedle patch kit according to another embodiment of the present disclosure.

FIG. 7 is a schematic diagram illustrating a cross-section when the microneedle patch shown in FIG. 1 is attached to a protruding affected area.

FIG. 8 is a confocal image measuring fluorescent material delivery distribution of the microneedle patch shown in FIG. 1.

### BEST MODE FOR INVENTION

The microneedle patch with improved deformability of the present disclosure and a microneedle patch kit including the same will be described in detail. The terms used in this specification are general terms that are currently widely used as much as possible while considering the function of the present disclosure, but this may vary depending on the intention or precedent of a technician working in the related field, the emergence of new technology, and the like. Unless otherwise defined, the technical and scientific terms used may have meanings commonly understood by those skilled in the art in the technical field to which this invention belongs.

As used in this specification and the appended claims, singular expressions include plural expressions, unless the context clearly dictates the singular. In addition, plural expressions include singular expressions, unless the context clearly specifies plural expressions.

In this specification and the appended patent claims, terms such as “include” or “have” mean the presence of features or components described in the specification, and, unless specifically limited, the terms do not preclude the possibility of adding one or more other features or components.

In addition, the numerical range used in this specification includes a lower limit and an upper limit and all values within the range, increments logically derived from the shape and width of the defined range, all double-defined values, and all possible combinations of the upper and lower limits of a numerical range defined in different forms. Unless otherwise specified in the specification of the present disclosure, values outside the numerical range that may occur due to experimental error or rounding of values are also included in the defined numerical range.

The terms “about” and the like used in this specification and the appended claims are used to encompass tolerance when tolerance exists.

Conventionally, a microneedle patch including a plurality of microneedles have poor flexibility, so when the microneedle patch is attached to a protruding and curved affected area, only a portion of the microneedles are inserted into the affected area, so that there is a problem in that an amount of drug delivered to the affected area is reduced, which reduces the treatment effect. In addition, when attaching a microneedle patch to a face with active movement, there is a problem in which the microneedle patch is easily detached from the skin due to low adhesion.

Accordingly, after in-depth research, the present applicant has developed a microneedle patch providing excellent drug delivery and inflammation relief effects, since microneedles may be uniformly inserted into the central portion and the peripheral portion of the protruding affected area through a microneedle path with high deformability, thereby improving adhesion.

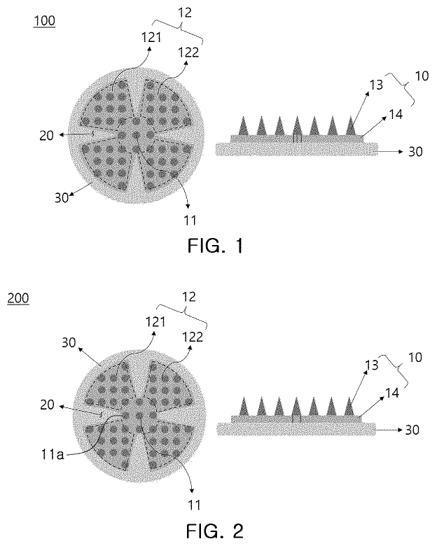

FIG. 1 illustrates a microneedle patch 100 according to an embodiment of the present disclosure.

Referring to FIG. 1, the microneedle patch 100 according to the present disclosure may include a hard substrate 14, and a microneedle array 10 including a plurality of microneedles 13 disposed on the hard substrate 14; and a soft substrate disposed below the microneedle array.

The microneedle array 10 includes a central portion 11 and a plurality of peripheral portions 12 connected to the central portion 11 and surrounding the central portion 11. The peripheral portions 121 and 122 among the peripheral portions 12, adjacent to each other, are disposed to be spaced from each other and are folded through a gap 20 between the adjacent peripheral portions 121 and 122.

As shown in FIG. 1, deformability of the microneedle patch 100 may be improved by providing a gap 20 between the peripheral portion 121 of the microneedle array 10 and the adjacent peripheral portion 122. When the microneedle patch 100 is attached to a curved affected area, the microneedle array 10 having low flexibility may be easily folded due to the gap 20, so that adhesion may be improved. Since the microneedle array 10 may be folded and attached to the affected area with high deformability, the number of microneedles 13 inserted into the protruding affected area may be increased. Accordingly, an effective ingredient contained in the microneedle array 10 may be uniformly delivered to the entire affected area through the microneedle 13, thereby improving a treatment effect.

An active ingredient is an ingredient exerting a pharmacological action to provide direct or indirect effects, and the active ingredient may provide sebum secretion reduction, antibacterial, anti-inflammatory, and wound healing effects. When the microneedle 13 is inserted into the affected area, the active ingredient may be quickly delivered to a local area through the microneedle 13, so the microneedle patch with an excellent treatment effect on the affected area may be manufactured.

In an embodiment, the number of peripheral portions 20 may be 2 to 20 or 2 to 10, and preferably 2 to 6. In the above range, the microneedle patch 100 may exhibit high deformability and can cover the affected area with excellent adhesion, but an embodiment of the present disclosure is not limited thereto.

An area of the peripheral portion 12 may be larger than an area of the gap 20 located between the adjacent peripheral portion 12. Since the area of the peripheral portion 12 is larger than the area of the gap 20, it is advantageous because the microneedle 13 may be inserted as a whole while surrounding the peripheral portion of the affected area. The area of the peripheral portion 12 refers to an area defined by being separated from the central portion thereof by a straight line obtained by connecting an innermost end portion of the gap 20 to an innermost end portion of the adjacent gap 20. In addition, the area of the gap 20 refers to the area of a closed curve formed by connecting a shortest distance obtained by connecting an outermost circumferential surface of the peripheral portion 12 and an outermost peripheral surface of the adjacent peripheral portion 12 with a straight line.

As shown in FIG. 1, flexibility may be provided to the microneedle patch 100 by disposing a soft substrate 30 below the microneedle array 10. In addition, the upper surface of the soft substrate 30 may have adhesion, so that the microneedle patch 100 including the microneedle array 10 with low flexibility may be attached to the curved affected area for a long time.

The gap 20 may be a planar portion having a step difference from the hard substrate 14 of the microneedle array 10, and may be formed by exposing an upper surface of the soft substrate 30. The soft substrate 30 having adhesion may be exposed to the gap 20 located between peripheral portion 12 adjacent to the microneedle patch 100, thereby improving adhesion of the microneedle patch 100.

Specifically, the central portion 11 of the microneedle array 10 may be in contact with a protruding affected area, and the peripheral portion 12 of the microneedle array 10 may surround the peripheral portion of the affected area and be in contact therewith. As the number of microneedles 13 inserted into the affected area increases, an effective ingredient delivery effect and an affected area treatment effect through the microneedle 13 may be improved.

In particular, the central portion 11 of the microneedle array 10 includes a microneedle 13, and the protruding central portion of the affected area may be in contact with the central portion 11 of the microneedle array 10 and the microneedle 13 may be inserted into the central portion of the affected area. An effect of relieving inflammation in the affected area may be further improved by quickly adsorbing an exudate, such as ooze, pus, and the like, through the microneedle 13 inserted into the central portion of the affected area with a high discharge amount of the exudate.

The plurality of microneedles 13 may be disposed to be spaced apart on the hard substrate 14. Specifically, the microneedles 13 may have a needle-shaped structure of which a width thereof decreases away from the hard substrate 14, and may be inserted into the affected area through a sharp tip located at an end of the microneedle 13. Without limitation, a length of the microneedle 13 may be 100 to 1000 μm or 200 to 800 μm, and may be substantially 300 to 600 μm.

FIG. 2 illustrates a microneedle patch 200 according to another embodiment of the present disclosure.

Referring to FIG. 2, an outer peripheral surface 11a of the central portion 11 of the microneedle patch 200, in contact with the gap 20 may be rounded. When the microneedle patch 200 or the microneedle patch 200 are used, the problem in which the hard microneedle array 10 is split and damaged may be minimized.

According to a non-limiting embodiment, according to the method of manufacturing the microneedle path 200, a microneedle array 10 in a shape corresponding to the central portion 11 may be manufactured, and then a portion of the microneedle array 10 may be cut in a direction from the outer peripheral surface of the central portion 11 to the outer peripheral surface of the microneedle array 10, to form a gap 20. As a more specific example, when manufacturing the microneedle patch 100 having a circular central portion 11, a circular microneedle array 10 including a hard substrate 14 and a plurality of microneedles 13 located on the hard substrate 14 may be manufactured using a mold, and then a portion of the circular microneedle array 10, excluding the central portion 11 may be cut with a blade or a laser to form a gap 20 and a plurality of peripheral portions 12. The outer peripheral surface of the central portion 11, in contact with the gap 20 may be sharply cut into a V shape, or cut into a U shape and rounded to manufacture the microneedle patch 100.

According to another non-limiting embodiment, a pattern corresponding to the microneedle array 10 in a mold, so that the microneedle patch 100 may be manufactured including a gap 20 between the peripheral portion 121 and the adjacent peripheral portion 122. Specifically, the mold may include a plurality of engraved grooves corresponding to the microneedles 13 and embossed patterns corresponding to the gap 20. After forming the microneedle array 10 using the mold and attaching the soft substrate 30 to the microneedle array 10, the microneedle array 10 may be separated from the mold, so that a microneedle patch 100 including the gap 20 between the peripheral portion 121 and the adjacent peripheral portion 122 may be manufactured.

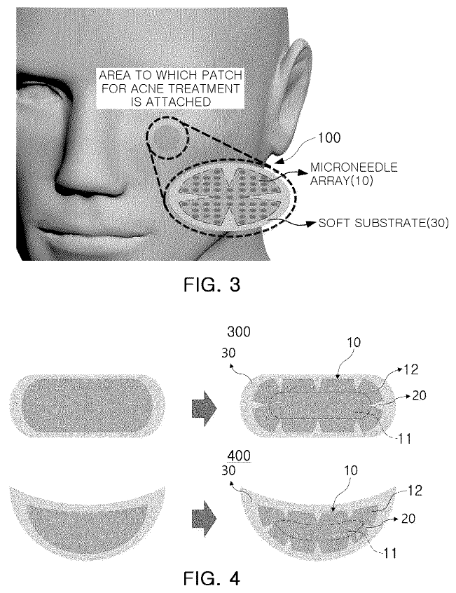

FIG. 3 illustrates a schematic diagram of attaching a microneedle patch 100 according to an embodiment of the present invention to an affected area of the facial skin of a human body.

Referring to FIG. 3, an upper surface of the microneedle patch 100 of the present disclosure on which the microneedle array 10 is formed is in contact with the skin of the human body, and the microneedle is inserted into an affected area and attached. In this case, a lower surface of the microneedle patch 100 on which the soft substrate 30 is disposed is exposed to the outside. When attaching the microneedle patch 100 to the affected area, the microneedle array 10 may be deformed into a shape corresponding to a surface of the affected area as a gap 20 is formed, and may come into close contact with the affected area. That is, the gap 20 may function as a notch of the microneedle array 10.

As shown in FIG. 3, the upper surface of the microneedle patch 100, in contact with the affected area has both a region in which the microneedle array 10 is disposed and a region of the soft substrate 30 exposed by the gap 20 of the microneedle array 10. In addition, in order to increase the adhesion of the microneedle patch 100 to the affected area, as the soft substrate 30 having an adhesive upper surface has a larger area than the microneedle array 10, the adhesive upper surface of the soft substrate may be partially exposed along an edge of the microneedle array 10.

FIG. 4 illustrates microneedle patches 300 and 400 according to another embodiment of the present disclosure, illustrating a central portion 11 of various shapes and a peripheral portion 12 according to the central portion 11.

The central portion 11 of the microneedle patch 100 is not particularly limited as long as it has a shape that can be attached to the affected area. The central portion 11 of the microneedle patch 100 may be planar with curved edges, such as a circle or oval. Alternatively, the central portion may be planar with straight edges, such as a triangle, square, or rectangle. Alternatively, as shown in FIG. 4, the central portion may planar with both straight and curved edges. That is, the shape of the microneedle patch 100 is not particularly limited as long as the central portion 11 and the peripheral portion 12 are formed, and adhesion may be improved by adopting a microneedle patch 100 having the central portion 11 and the peripheral portion 12 of an appropriate shape depending on the position to which the microneedle patch 100 is attached.

In order to prevent the unused microneedle patch 100 from being exposed to contaminants, a protective film 40 disposed on the microneedle patch 100 may be further included. The microneedle patch 100 may be used more hygienically by preventing external contaminants, such as foreign substances, bacteria, viruses, and the like, from penetrating into the microneedle patch 100 and preventing the risk of secondary infection due to contamination of the affected area.

In an embodiment, the soft substrate 30 may include a hydrocolloid layer. The soft substrate 30 including a hydrocolloid layer may seal the affected area to prevent foreign substances such as bacteria from penetrating into the affected area, and can protect and seal the affected area by absorbing an exudate.

In an embodiment, the microneedle array 10 may contain a biocompatible polymer. The biocompatible polymer may include hyaluronic acid, lipids, chitosan, protein, DNA, collagen, alginic acid, gelatin, silk fibroin, poly (lactic-co-glycolic acid) (PLGA), polylactic acid (PLA), polyvinyl polypyrrolidone (PVP), polyvinyl alcohol, PVA), sucrose, maltose, polyethylene, SU-8, polymethylmethacrylate (PMMA), polycarbonate, polymethyl vinyl ether-co-maleic anhydride (PMVE/MA) , sodium chondroitin sulfate, polycaprolactone (PCL), poly (ethylene glycol), poly acrylic acid (PAA), poly-γ-glutamic acid, and the like, and may preferably include hyaluronic acid. As a specific example, the microneedles 13 and the hard substrate 14 included in the microneedle array 10 may include the same or different materials, and it is preferable that the microneedle 13 and the hard substrate 14 include the same materials.

The present disclosure provides a microneedle patch kit 1 including the above-described microneedle patch 100.

The microneedle patch kit 1 according to the present disclosure includes a release film 50; and a plurality of microneedle patches 100 disposed to be spaced apart from each other on the release film 50, and since the microneedle patch is the same as described above, detailed description thereof will be omitted.

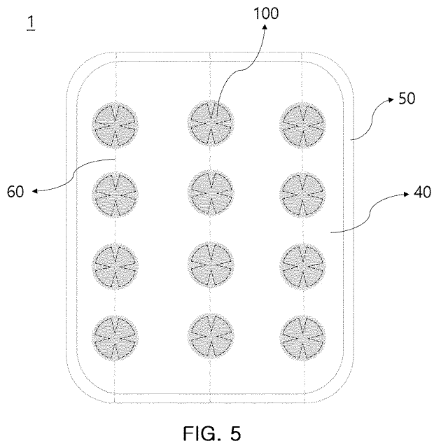

As shown in FIG. 5, the release film 50 may include a fracture line 60 formed in one direction, and a plurality of microneedle patches 100 may be disposed on the fracture line 60, so that the microneedle patches 100 can be separated from the release film 50 and be used conveniently. When removing the microneedle patch 100 from the release film 50, the microneedle patch 100 may not be in contact with contaminated hands. By preventing contamination of the patch, infection of an affected area caused by the microneedle patch 100 can be prevented.

In order to prevent the microneedle patch 100 from being exposed to contamination, a protective film 40 covering and protecting the release film 50 and the plurality of microneedle patches 100 may be further included. As described above, the microneedle patch 100 may be hygienically and safely protected from external contaminants such as foreign substances, bacteria, and viruses through the protective film 40.

In an embodiment, the release film 50 and the protective film 40 may include a transparent plastic film such as polyethylene terephthalate (PET), polypropylene (PP), polyethylene (PE) or the like, and may preferably include polyethylene terephthalate (PET), but the present disclosure is not limited thereto.

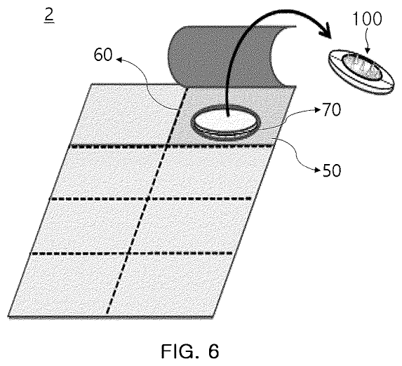

In another aspect of the present disclosure, referring to FIG. 6, the microneedle patch kit 2 may further include a plurality of microneedle patch receiving portions 70 disposed to be spaced apart from the fracture line 60, and a plurality of microneedle patches 100 may be individually packaged by the fracture line 60. The microneedle patch kit 2 in which the microneedle patch 100 is individually packaged is sealed through the recessed microneedle patch receiving portion 70, so that the unused microneedle patch 100 is not exposed to external environments, which is advantageous since penetration of contaminants and penetration of moisture may be prevented.

Specifically, the individually packaged microneedle patch 100 has a release paper attached to an upper surface of the soft substrate 30, that is, an adhesive surface thereof, so that the microneedle patch 100 may be used hygienically while maintaining adhesion of the soft substrate 30. The release paper has a separation line cut in one direction, so that the release paper may be easily separated from the soft substrate 30 when using the microneedle patch 100.

Hereinafter, the present disclosure will be described in more detail through Examples.

### EXAMPLE 1

A mold including a plurality of engraved grooves corresponding to microneedles was manufactured by molding polydimethylsiloxane (PDMS) on an aluminum master mold. Thereafter, hyaluronic acid was molded into a PDMS mold and cured to prepare a microneedle array. A hydrocolloid layer was attached to the microneedle array as a soft substrate. In this case, an adhesive surface of the hydrocolloid layer was disposed to be in contact with the microneedle array. Thereafter, the microneedle array including a soft substrate was separated from the PDMS mold, and a peripheral portion of the microneedle array was cut with a laser to form a gap to manufacture a microneedle patch as shown in FIG. 1.

### EXAMPLE 2

A microneedle patch was manufactured in the same manner as Example 1, except that, when cutting the microneedle array, the microneedle array was cut using a blade.

### EXAMPLE 3

A microneedle patch was manufactured in the same manner as in Example 1, except that, when cutting the microneedle array, an outer circumferential surface of a center portion, in contact with the gap was rounded, to manufacture a microneedle patch as shown in FIG. 2.

### Comparative Example 1

A microneedle patch was manufactured in the same manner as Example 1, except that the microneedle array was not cut.

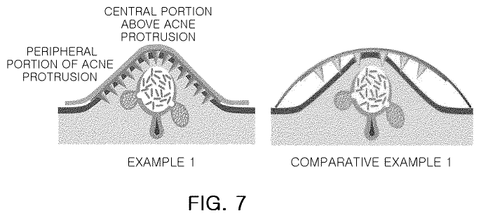

FIG. 7 is a cross-sectional view illustrating a cross-section of the microneedle patch manufactured by the method in Example 1 and Comparative Example 1, when attached to a protruding trouble. The microneedle patch of Example 1 was easily folded through a gap between a peripheral portion and an adjacent peripheral portion. Deformability of the microneedle patch was improved so that not only were microneedles also inserted into the central portion of the affected area, but microneedle patches were uniformly inserted into the peripheral portion thereof. However, the microneedle patch of Comparative Example 1 did not have a space for a microneedle array to be folded, so deformability was deteriorated and only a portion of the microneedle included in the microneedle array was inserted into the affected area, and the microneedle could not be inserted into the peripheral portion of the affected area, so that adhesion was deteriorated.

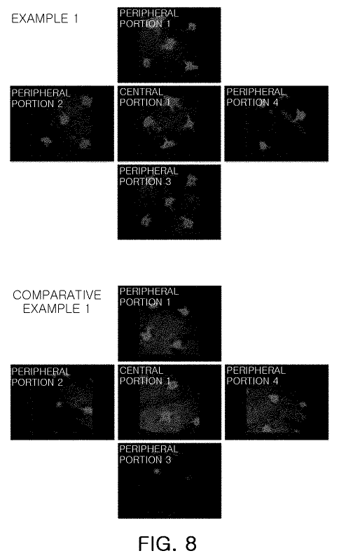

FIG. 8 is an image evaluating a drug delivery effect of the microneedle patch manufactured by the method of Example 1 and Comparative Example 1. A microneedle patch injected with rhodamine B, a fluorescent dye, was attached to pig skin simulating a protruding affected area, and then the effects were compared through confocal imaging.

As shown in FIG. 8, in the case of the microneedle patch of Comparative Example 1, a fluorescent material was delivered only to a portion of the central portion and the peripheral portions of the protruding affected area, so it can be seen that the drug was delivered non-uniformly to the protruding area. On the other hand, when the microneedle patch of Example 1 was attached to the protruding affected area, the fluorescent material was delivered uniformly to the central and peripheral regions of the affected area. All microneedles included in the microneedle array were uniformly inserted into the affected area by being in close contact with not only the central portion of the protruding affected area but also the peripheral portion thereof. By improving the deformability of the microneedle patch, it can be confirmed that the microneedle patch is implemented to effectively deliver the active ingredient to the local area by uniformly inserting the microneedle into the central portion and the peripheral portion of the affected area.

As described above, the present disclosure has been described with specific details, limited embodiments, and drawings, but these are provided only to facilitate a more general understanding of the present disclosure, and the present invention is not limited to the above embodiments, and various modifications and variations may be made from these descriptions by those skilled in the art.

Accordingly, the spirit of the present disclosure should not be limited to the described embodiments, and the scope of the patent claims described below as well as all modifications that are equivalent or equivalent to the scope of this patent claim shall fall within the scope of the spirit of the present disclosure.

1. A microneedle patch, comprising:
a microneedle array including a hard planar substrate, and a plurality of microneedles disposed on the hard substrate,
wherein the microneedle array includes a central portion and a plurality of peripheral portions connected to the central portion and surrounding the central portion,
wherein the peripheral portion is disposed to be spaced apart from an adjacent peripheral portion, and is folded through a gap between the adjacent peripheral portion.

2. The microneedle patch of claim 1, further comprising:
a soft substrate disposed below the microneedle array.

3. The microneedle patch of claim 1, wherein the gap is a planar portion having a step difference from the microneedle array.

4. The microneedle patch of claim 2, wherein the gap is formed by exposing an upper surface of the soft substrate.

5. The microneedle patch of claim 2, wherein the upper surface of the soft substrate has adhesion.

6. The microneedle patch of claim 1, wherein an area of the peripheral portion is greater than an area of the gap between the adjacent peripheral portion.

7. The microneedle patch of claim 1, wherein an outer peripheral surface of the central portion, in contact with the gap is rounded.

8. The microneedle patch of claim 1, further comprising:
a protective film disposed on the microneedle patch.

9. The microneedle patch of claim 5, wherein the soft substrate comprises a hydrocolloid layer.

10. The microneedle patch of claim 1, wherein the microneedle array contains a biocompatible polymer.

11. The microneedle patch of claim 1, wherein the microneedle array contains an active ingredient.

12. The microneedle patch of claim 1, wherein the central portion is in contact with a protruding affected area, and the peripheral portion surrounds a peripheral portion of the affected area and is in contact therewith.

13. A microneedle patch kit, comprising:
a release film; and
a plurality of microneedle patches disposed to be spaced apart from each other on the release film,
wherein the microneedle patch includes the microneedle patch according to claim 1.

14. The microneedle patch kit of claim 13, further comprising:
a protective film covering and protecting the release film and the plurality of microneedle patches.

15. The microneedle patch kit of claim 13, wherein the release film comprises a fracture line formed in one direction.

16. The microneedle patch kit of claim 15, wherein the plurality of microneedle patches are disposed on the fracture line.

17. The microneedle patch kit of claim 15, wherein the release film further comprises a plurality of microneedle patch receiving portions disposed to be spaced apart from the fracture line,
wherein the plurality of microneedle patches are individually packaged by the microneedle patch receiving portions.
